(19) United States
(12) Patent Application Publication
(10) Pub. No.: US 2007/0118194 A1
(43) Pub. Date: May 24, 2007

(54) Non-ambient temperature therapy system with automatic treatment temperature maintenance

(75) Inventors: Bradley R. Mason, Rancho Santa Fe, CA (US); Jeffrey T. Mason, Escondido, CA (US)

Correspondence Address: RODNEY F. BROWN, 3365 BALTIMORE STREET, SAN DIEGO, CA, 92117, US

(73) Assignee: Breg, Inc.

(21) Appl. No.: 11/285,827

(22) Filed: Nov. 22, 2005

(51) Int. Cl. A61F 7/00

(52) U.S. Cl. 607/104; 607/108

(57) ABSTRACT

A therapy system for therapeutically treating a desired region of the body includes a fluid reservoir, a heat transfer pad, and first and second fluid circulation lines. The heat transfer pad includes a first pad port, a bladder, and a second pad port. The first fluid circulation line is connectable to the first pad port for withdrawing heat transfer fluid from the fluid reservoir and delivering it to the heat transfer pad. The second fluid circulation line is connectable to the second pad port for withdrawing heat transfer fluid from the heat transfer pad and delivering it to the fluid reservoir. The heat transfer pad additionally includes a pad flowpath adjustment member positioned in the pad flowpath to adjust the system resistance to flow. The adjusted system resistance to flow fixes the desired treatment temperature of the heat transfer pad in response to the heat transfer fluid circulating through the pad flowpath. The desired treatment temperature is correlated to the body region being treated.

### TECHNICAL FIELD

The present invention relates generally to a system for treating bodily injuries and ailments by cooling or heating the affected region of the body, and more particularly, to a non-ambient temperature therapy system which circulates a heat transfer fluid through a heat transfer pad mounted on the affected region of the body of a patient.

### BACKGROUND OF THE INVENTION

Bodily injuries and ailments are commonly treated by applying a non-ambient temperature material to the affected region of the body. For example, a low temperature material, typically applied in the form of cold water, ice or a cold pack, may advantageously inhibit swelling in the region of the injury. A high temperature material, typically applied in the form of hot water, a hot pack or an active heating element, may advantageously reduce pain and promote healing. A number of splint devices are known in the art for applying non-ambient temperature materials to injured or otherwise ailing regions of the body as evidenced by U.S. Pat. No. 3,548,819 to Davis et al; U.S. Pat. No. 3,901,225 to Sconce; and U.S. Pat. No. 4,706,658 to Cronin. One disadvantage of such devices is that the low temperature materials become warmer as they remain in contact with the body during treatment and the body transfers heat to the low temperature materials. Conversely, high temperature materials become cooler as they transfer heat to the body. This disadvantage can be remedied by periodically replacing the non-ambient temperature materials. However, constant replenishment of these materials is cumbersome and inconvenient, and results in periodic treatment temperature fluctuations.

In response to this problem, a number of systems have been developed for continuously circulating a cooling fluid from a low temperature reservoir to a desired body location. Such systems are typified by U.S. Pat. No. 2,726,658 to Chessey; U.S. Pat. No. 3,683,902 to Artemenko et al; and U.S. Pat. No. 4,962,761 to Golden. These fluid circulation systems in general are relatively complex, rendering them costly to manufacture and maintain, as well as difficult to operate. Accordingly, the systems are not practical for widespread use.

U.S. Pat. No. 5,241,951 to Mason et al discloses a therapeutic treatment system which rectifies the shortcomings of the above-referenced fluid circulation systems. The therapeutic treatment system of U.S. Pat. No. 5,241,951 is relatively simple, rendering it less costly to manufacture and maintain and enabling greater ease of operation than the prior systems. The system of U.S. Pat. No. 5,241,951 consists essentially of a fluid reservoir, a submersible single-speed pump, a pad having an internal pad flowpath, inlet and outlet lines connecting the pad flowpath to the pump and a user adjustable in-line flow control valve. The system is operated by filling the reservoir with a non-ambient temperature treatment fluid and submersing the pump in the fluid. The pad is positioned on the desired treatment region of a patient and the pump is activated to deliver fresh treatment fluid from the reservoir to the pad flowpath via the inlet line and return spent treatment fluid from the pad flowpath to the reservoir via the outlet line. The patient regulates the treatment temperature of the pad by manually adjusting the flow control valve to control the flow rate of fluid through the pad flowpath.

The system of U.S. Pat. No. 5,241,951 has been shown to provide effective therapeutic treatment to the body. Nevertheless, it has been found that the patient is not always adept at properly adjusting the flow control valve to achieve a desired treatment temperature in the pad. In the case of low temperature treatment, if the patient sets the flow rate of the fluid through the pad too high, the pad may become too cold and harm the treatment region of the body. Conversely if the patient sets the flow rate too low, the pad may not become cold enough and the low temperature treatment will not be effective. In the case of high temperature treatment, if the patient sets the flow rate too high, the pad may become too hot, similarly harming the treatment region, while setting the flow rate too low, may similarly prevent the pad from becoming hot enough, rendering the high temperature treatment ineffective.

The present invention recognizes the need for an improved fluid circulation-type therapeutic treatment system employing a non-ambient temperature pad which obviates the need for user interface during operation while providing effective treatment of the patient. Accordingly, it is generally an object of the present invention to provide an improved non-ambient temperature therapy system, which is effective, safe and reliable. More particularly, it is an object of the present invention to provide such an improved non-ambient temperature therapy system, which automatically maintains the treatment temperature in the pad at a desired level in the absence of user interface. It is another object of the present invention to provide such an improved non-ambient temperature therapy system, which includes a family of pads, each pad having a different size or shape specific to a different application of the system. It is still another object of the present invention to provide such an improved non-ambient temperature therapy system, wherein all of the alternately sized or shaped pads of the pad family can be used interchangeably within the system in response to the desired application of the system. It is still another object of the present invention to provide such an improved non-ambient temperature therapy system, which enables substitution of any of the alternately sized or shaped pads for another within the system without disrupting the automatic treatment temperature maintenance function of the system. These objects and others are achieved in accordance with the invention described hereafter.

### SUMMARY OF THE INVENTION

The present invention is a therapy system for therapeutically treating a desired region of the body which comprises a fluid reservoir, a heat transfer pad, first and second fluid circulation lines, and a pump. The fluid reservoir stores a charge of a heat transfer fluid at a non-ambient temperature. The heat transfer pad is conformable to a body region which has an ambient temperature and the non-ambient temperature of the heat transfer fluid is either a lower temperature or a higher temperature than the ambient temperature of the body region.

The heat transfer pad includes a first pad port, a bladder, and a second pad port, which define a pad flowpath extending in series therethrough. The first fluid circulation line is connectable to the first pad port and is in fluid communication with the fluid reservoir to withdraw the heat transfer fluid from the fluid reservoir and deliver the heat transfer fluid to the heat transfer pad via the first pad port. The second fluid circulation line is connectable to the second pad port and is in fluid communication with the fluid reservoir to withdraw the heat transfer fluid from the heat transfer pad via the second pad port and deliver the heat transfer fluid to the fluid reservoir. The first fluid circulation line, pad flowpath, and second fluid circulation line define a system flowpath extending in series therethrough.

The pump includes a motor fixed at a set operating speed which defines a maximum pump output. The pump is in fluid communication with the fluid reservoir and the first fluid circulation line to drive the heat transfer fluid through the system flowpath against a system resistance to flow. The pump preferably operates at an actual pump output less than the maximum pump output. The heat transfer pad additionally includes a pad flowpath adjustment member positioned in the pad flowpath, preferably mounted in the second pad port, which adjusts the system resistance to flow to an adjusted system resistance to flow. The adjusted system resistance to flow fixes a desired treatment temperature of the heat transfer pad in response to the heat transfer fluid circulating through the pad flowpath at a pad flow rate. The adjusted system resistance to flow is preferably greater than the system resistance to flow and the desired treatment temperature of the heat transfer pad is correlated to the body region being treated and is preferably indirectly correlated with the adjusted system resistance to flow.

In accordance with one embodiment, the pad flowpath adjustment member is a threaded rod positioned in the first or second pad port. In accordance with another embodiment, the pad flowpath adjustment member is a crimp formed in a wall of the first or second pad port. In accordance with yet another embodiment, the pad flowpath adjustment member is a plate or nozzle having an aperture formed therethrough positioned in the first or second pad port. In accordance with still another embodiment, the pad flowpath adjustment member is a tubing segment of the second pad port having a reduced cross-sectional area relative to the first pad port or the pad flowpath adjustment member is a tubing segment of the first pad port having a reduced cross-sectional area relative to the second pad port.

The present invention is alternately a therapy system for therapeutically treating a desired region of the body which comprises a fluid reservoir, a pad family, first and second fluid circulation lines, and a pump. The fluid reservoir, first and second fluid circulation lines and pump are all essentially as described above. The pad family includes a first and a second heat transfer pad. The first heat transfer pad has a first geometry and is conformable to a first body region having an ambient temperature. The first heat transfer pad includes a first pad port, a bladder, and a second pad port and defines a first pad flowpath extending in series therethrough. The second heat transfer pad has a second geometry different from the first geometry and is conformable to a second body region having the ambient temperature. The second heat transfer pad includes a first pad port, a bladder, and a second pad port and defines a second pad flowpath extending in series therethrough.

The first fluid circulation line is connectable to the respective first pad port of the first or second heat transfer pad and is in fluid communication with the fluid reservoir to withdraw the heat transfer fluid from the fluid reservoir and deliver the heat transfer fluid to the first or second heat transfer pad via the respective first pad port. The second fluid circulation line is connectable to the respective second pad port of the first or second heat transfer pad and is in fluid communication with the fluid reservoir to withdraw the heat transfer fluid from the first or second heat transfer pad via the respective second pad port and deliver the heat transfer fluid to the fluid reservoir. The first fluid circulation line, the first pad flowpath, and the second fluid circulation line define a first system flowpath extending in series therethrough. The first fluid circulation line, the second pad flowpath, and the second fluid circulation line alternately define a second system flowpath extending in series therethrough.

The pump is in fluid communication with the fluid reservoir and the first fluid circulation line to drive the heat transfer fluid through the first or second system flowpath against a system resistance to flow. A first pad flowpath adjustment member is positioned in the first pad flowpath, preferably mounted in the second pad port of the first heat transfer pad, which adjusts the system resistance to flow to a first adjusted system resistance to flow. The first adjusted system resistance to flow fixes a first desired treatment temperature of the first heat transfer pad in response to the heat transfer fluid circulating through the first pad flowpath at a first pad flow rate. The first desired treatment temperature is correlated to the first body region. A second pad flowpath adjustment member is positioned in the second pad flowpath, preferably mounted in the second pad port of the second heat transfer pad, to adjust the system resistance to flow to a second adjusted system resistance to flow. The second adjusted system resistance to flow fixes a second desired treatment temperature of the second heat transfer pad in response to the heat transfer fluid circulating through the second pad flowpath at a second pad flow rate. The second desired treatment temperature is correlated to the second body region.

In a preferred case, the second desired treatment temperature is less than the first desired treatment temperature. In accordance with this case, a preferred first body region is an ankle and a preferred second body region is a knee.

The present invention is alternately a method for therapeutically treating a desired region of the body with a non-ambient temperature treatment fluid. A charge of a heat transfer fluid is stored at a non-ambient temperature in a fluid reservoir. A heat transfer pad is positioned on a body region having an ambient temperature. The heat transfer pad includes a first pad port, a bladder, and a second pad port and defining a pad flowpath extending in series therethrough. A first fluid circulation line in fluid communication with the fluid reservoir is connected to the first pad port. The heat transfer fluid is withdrawn from the fluid reservoir via the first fluid circulation line and delivered to the heat transfer pad via the first pad port. A second fluid circulation line in fluid communication with the fluid reservoir is connected to the second pad port. The first fluid circulation line, pad flowpath, and second fluid circulation line define a system flowpath extending in series therethrough. The heat transfer fluid is withdrawn from the heat transfer pad via the second pad port and delivered to the fluid reservoir via the second fluid circulation line.

A pump in fluid communication with the fluid reservoir and the first fluid circulation line drives the heat transfer fluid through the system flowpath against a system resistance to flow. The pump includes a motor fixed at a set operating speed which defines a maximum pump output. A pad flowpath adjustment member is positioned in the pad flowpath, preferably mounted in the second pad port, which adjusts the system resistance to flow to an adjusted system resistance to flow. The adjusted system resistance to flow fixes a desired treatment temperature of the heat transfer pad in response to the heat transfer fluid circulating through the pad flowpath at a pad flow rate. The desired treatment temperature is correlated to the body region.

In accordance with a preferred embodiment, the body region is a first body region, the heat transfer pad is a first heat transfer pad, the pad flowpath is a first pad flowpath, the system flowpath is a first system flowpath, the pad flowpath adjustment member is a first pad flowpath adjustment member, the adjusted system resistance to flow is a first adjusted system resistance to flow, the desired treatment temperature is a first desired treatment temperature, and the pad flow rate is a first pad flow rate. The method further comprises positioning a second heat transfer pad on a second body region having the ambient temperature. The second heat transfer pad includes a first pad port, a bladder, and a second pad port and defines a second pad flowpath extending in series therethrough;

The first fluid circulation line is disconnected from the first pad port of the first heat transfer pad and the first fluid circulation line is reconnected to the first pad port of the second heat transfer pad. The heat transfer fluid is withdrawn from the fluid reservoir via the first fluid circulation line and the heat transfer fluid is delivered to the second heat transfer pad via the first pad port of the second heat transfer pad. The second fluid circulation line is disconnected from the second pad port of the second heat transfer pad and the second fluid circulation line is reconnected to the second pad port of the second heat transfer pad. The first fluid circulation line, second pad flowpath, and second fluid circulation line define a second system flowpath extending in series therethrough. The heat transfer fluid is withdrawn from the second heat transfer pad via the second pad port of the second heat transfer pad and the heat transfer fluid is delivered to the fluid reservoir via the second fluid circulation line.

The pump drives the heat transfer fluid through the second system flowpath against the system resistance to flow. A second pad flowpath adjustment member is positioned in the second pad flowpath, preferably mounted in the second pad port of the second heat transfer pad, to adjust the system resistance to flow to a second adjusted system resistance to flow. The second adjusted system resistance to flow fixes a second desired treatment temperature of the second heat transfer pad in response to the heat transfer fluid circulating through the second pad flowpath at a second pad flow rate. The second desired treatment temperature is correlated to the second body region.

In a preferred case, the non-ambient temperature is a lower temperature than the ambient temperature of the first and second body regions, the second adjusted resistance to flow is less than the first adjusted system resistance to flow, and the second desired treatment temperature is less than the first desired treatment temperature. In accordance with this case, a preferred first body region is an ankle and a preferred second body region is a knee.

The present invention will be further understood from the drawings and the following detailed description.

### BRIEF DESCRIPTION OF THE DRAWINGS

FIG. 1 is an exploded view of a non-ambient temperature therapy system of the present invention in a disassembled state.

FIG. 2 is a perspective view of the non-ambient temperature therapy system of FIG. 1 in an assembled state, wherein the heat transfer pad of the system is operatively mounted on a patient.

FIG. 3 is a detailed plan view of the heat transfer pad included in the non-ambient temperature therapy system of FIGS. 1 and 2.

FIG. 4 is a cutaway side view of a pad flowpath adjustment member mounted in the flowpath of the heat transfer pad of FIG. 3.

FIG. 5 is a cutaway side view of an alternate embodiment of a pad flowpath adjustment member mounted in the flowpath of the heat transfer pad of FIG. 3.

FIG. 6 is a top view of the pad flowpath adjustment member of FIG. 5.

FIG. 7 is a cutaway side view of an alternate embodiment of a pad flowpath adjustment member mounted in the flowpath of the heat transfer pad of FIG. 3.

FIG. 8 is a top view of the pad flowpath adjustment member of FIG. 7.

FIG. 9 is a plan view of a pad family including a plurality of separate individual heat transfer pads, each having alternate utility in the non-ambient temperature therapy system of FIGS. 1 and 2.

### DESCRIPTION OF PREFERRED EMBODIMENTS

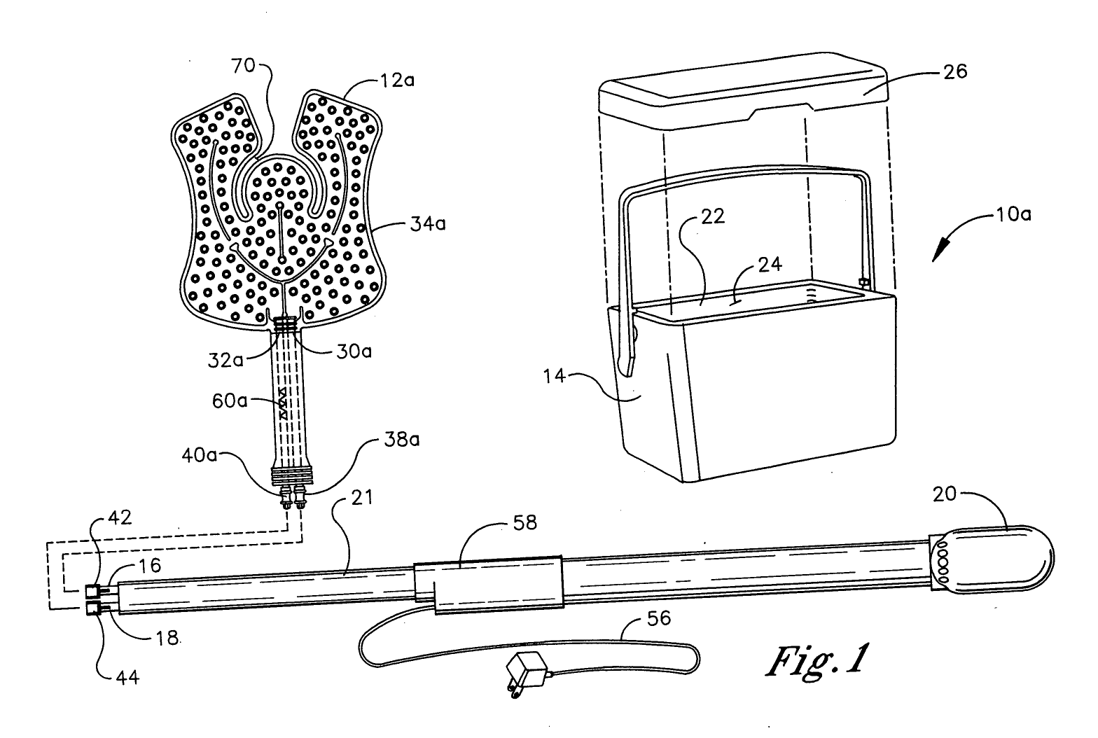

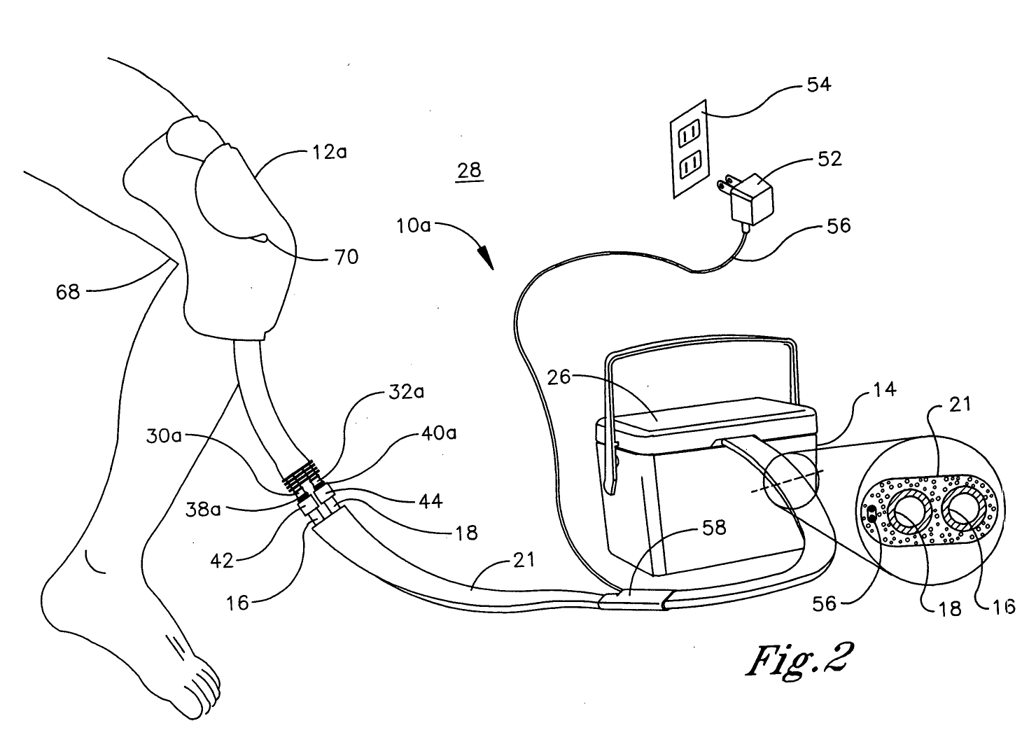

Referring initially to FIG. 1, a non-ambient temperature therapy system of the present invention is shown in a disassembled state and generally designated 10a. The non-ambient temperature therapy system 10a comprises a heat transfer pad 12a, a fluid reservoir 14, a first fluid circulation line 16, a second fluid circulation line 18, and a pump 20. Referring additionally to FIG. 2, the non-ambient temperature therapy system 10a is shown in an assembled state after a user has assembled the system 10a from the disassembled state. The user is typically a patient undergoing treatment with the non-ambient temperature therapy system 10a or a health care practitioner overseeing treatment of the patient with the system 10a. The term “non-ambient temperature” is used herein relative to the ambient body temperature of the patient. A high temperature treatment applies a non-ambient treatment temperature to the body which is greater than the ambient body temperature while a low temperature treatment applies a non-ambient treatment temperature to the body which is less than the ambient body temperature.

When the non-ambient temperature therapy system 10a is in the assembled state shown in FIG. 2, the heat transfer pad 12a and the fluid reservoir 14 are positioned a distance apart from one another with the first and second fluid circulation lines 16, 18 extending in parallel between them. The first fluid circulation line 16 functions as a pad inlet line, withdrawing a fresh non-ambient temperature heat transfer fluid from the fluid reservoir 14 and delivering the fresh non-ambient temperature heat transfer fluid to the heat transfer pad 12a in accordance with a preferred method of operation described hereafter. Conversely, the second fluid circulation line 18 functions as a pad outlet line, withdrawing the spent heat transfer fluid from the heat transfer pad 12a and delivering the spent heat transfer fluid to the fluid reservoir 14, likewise in accordance with the preferred method of operation described hereafter. The first and second fluid circulation lines 16, 18 are each preferably formed from a continuous length of identical flexible tubing each having the essentially same inside diameter.

An insulating line sheath 21 extends essentially the length of the first and second fluid circulation lines 16, 18 and covers the lines 16, 18, thereby enclosing the lines 16, 18 within a single integrated smooth tubular unit (as shown in the cross-section blow-up). The line sheath 21 is formed in its entirety from a supple material which renders the line sheath 21, in combination with the first and second fluid circulation lines 16, 18, fully flexible. The line sheath 21 has a durable exterior skin and an insulating foam interior which minimizes heat transfer between the first fluid circulation line 16 and the second fluid circulation line 18 or between the first and second fluid circulation lines 16, 18 and the surrounding environment 28. The line sheath 21 also prevents condensate formation on the exterior of the first and second fluid circulation lines 16, 18.

The fluid reservoir 14 is a hollow fluid container, which includes means for a user to access the interior of the container, thereby enabling the user to manually add the non-ambient temperature heat transfer fluid in bulk to the fluid reservoir 14 when charging the non-ambient temperature therapy system 10a or to manually withdraw heat transfer fluid in bulk from the fluid reservoir 14 when draining the system 10a. The fluid reservoir 14 is preferably a thermally-passive hollow fluid container having insulated walls 22 and a relatively wide accessible opening 24 at the top for addition or withdrawal of the heat transfer fluid. The preferred fluid reservoir 14 is additionally provided with a fitted removable lid 26, which enables the user to selectively cover the opening 24. Fitting the lid 26 across the opening 24 reduces the degree of heat transfer between the heat transfer fluid residing in the interior of the fluid reservoir 14 and the ambient atmosphere of the surrounding environment 28. The preferred fluid reservoir 14 described above is essentially the same or similar to a conventional picnic or beverage cooler.

The term “thermally-passive”, as used herein, characterizes a structure which is free of any active structural cooling or heating elements, such as refrigeration coils, heating coils, or the like, which act on the heat transfer fluid to actively cool or heat the fluid. The entirety of the non-ambient temperature therapy system 10a is likewise preferably characterized as thermally-passive insofar as the system 10a in its entirety is preferably free of any active structural cooling or heating elements. Notwithstanding the above, it is within the scope of the present invention to place a passive cooling medium in the fluid reservoir 14, such as ice or the like, to passively cool the heat transfer fluid therein.

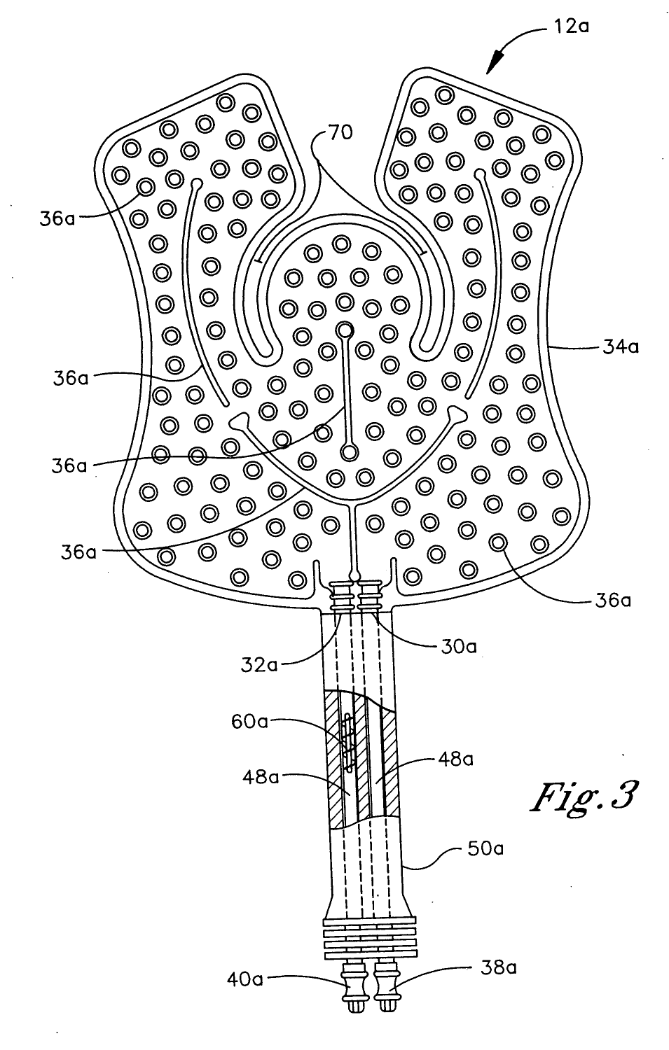

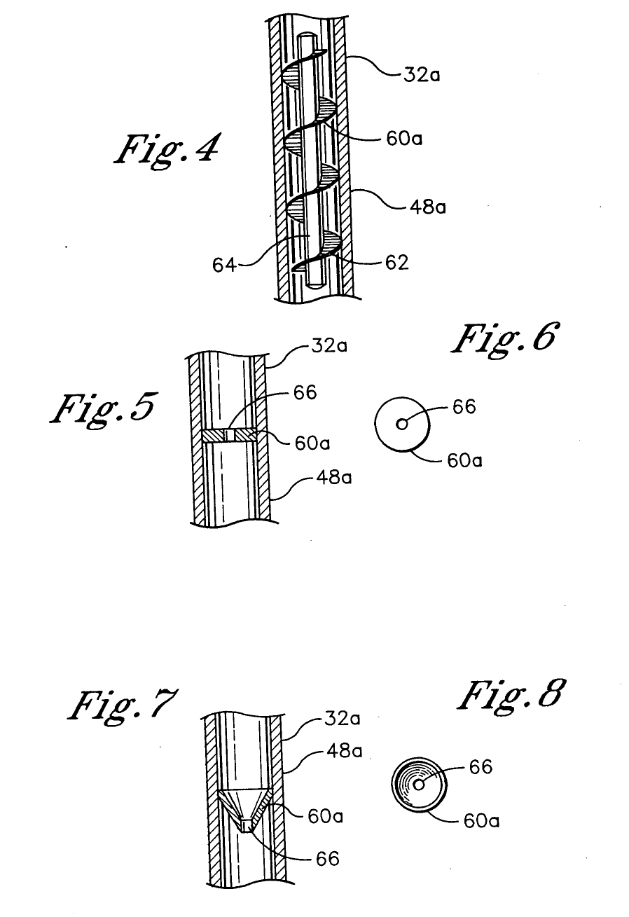

Referring additionally to FIG. 3, the heat transfer pad 12a includes a first pad port 30a which preferably functions as an inlet port, a second pad port 32a which preferably functions as an outlet port, and a bladder 34a which is positioned between the first and second pad ports 30a, 32a. The bladder 34a accommodates the first and second pad ports 30a, 32a and encloses a tortuous internal flowpath for the non-ambient temperature heat transfer fluid extending through the bladder 34a between the first and second pad ports 30a, 32a. In accordance with a preferred embodiment, the bladder 34a is an essentially planar member formed from a shaped upper sheet of a thin flexible heat-conductive material, such as a pliable polyurethane film, which is laid over an essentially identically shaped and dimensioned lower sheet of the same material. The upper and lower sheets are sealingly bonded together along their entire periphery with the exception of two distinctly separate relatively short segments of the periphery, which are maintained unbonded to provide the first and second pad ports 30a, 32a. The bladder 34a preferably has one or more flow diverters 36a integral therewith, which delineate the internal flowpath of the bladder 34a and enhance the tortuosity of the internal flowpath to facilitate distribution of the heat transfer fluid throughout the bladder 34a. The flow diverters 36a are preferably formed by periodically bonding the upper and lower sheets together at points interior to the periphery of the sheets.

Each first and second pad port 30a, 32a opens the internal flowpath of the bladder 34a to the surrounding environment 28. The first and second pad ports 30a, 32a include first and second port couplings 38a, 40a, which are positioned across the openings of the first and second pad ports 30a, 32a, respectively. The first and second port couplings 38a, 40a are configured to cooperatively and releasably mate with first and second line couplings 42, 44, respectively, which are included in the first and second fluid circulation lines 16,18, respectively, and are positioned across the open proximal ends of the first and second fluid circulation lines 16, 18. (The terms “proximal” and “distal” are used herein relative to the bladder 34a.) The couplings 38a, 40a, 42, 44 are preferably snap-action locking couplings which are selectively manually releasable.

The first and second port couplings 38a, 40a are male couplings and the first and second line couplings 42, 44 are female couplings. Although not shown, the first and second port couplings 38a, 40a are alternatively configured as female couplings and the first and second line couplings 42, 44 are configured as male couplings. Mating the first port coupling 38a with the first line coupling 42 and the second port coupling 40a with the second line coupling 44 forms a connective joint 46 between the heat transfer pad 12a and first and second fluid circulation lines 16, 18. The joint 46 provides fluid communication between the first and second pad ports 30a, 32a and the first and second fluid circulation lines 16, 18, respectively, as well as between the first and second fluid circulation lines 16, 18 and the internal flowpath of the bladder 34a which is integral with the first and second pad ports 30a, 32a.

In accordance with a preferred embodiment the first port coupling 38a and first line coupling 42 are uniquely and cooperatively configured so that the first port coupling 38a can only mate with the first line coupling 42 and not with the second line coupling 44. The second port coupling 40a and second line coupling 44 are likewise uniquely and cooperatively configured so that the second port coupling 40a can only mate with the second line coupling 44 and not with the first line coupling 42. As a result, the first pad port 30a can preferably only be coupled with the first fluid circulation line 16 and not with the second fluid circulation line 18. The second pad port 32a can preferably only be coupled with the second fluid circulation line 18 and not with the first fluid circulation line 16.

Each first and second port coupling 38a, 40a also includes an integral shutoff valve element which restricts access to the internal flowpath of the bladder 34a from the surrounding environment 28 via the first and second pad ports 30a, 32a when the first and second port couplings 38a, 40a are unmated. As such, the shutoff valve element is normally biased in the closed position by a cooperative biasing means, such as a spring or the like, when the first and second port couplings 38a, 40a are unmated. However, mating the first port coupling 38a with the first line coupling 42 and the second port coupling 40a with the second line coupling 44 brings the first and second line couplings 42, 44 into engagement with the shutoff valve element, thereby actively transitioning the shutoff valve element to the open position. When the shutoff valve element is in the open position, the shutoff valve element and associated biasing means do not substantially impede flow through the first and second port couplings 38a, 40a.

Each first and second pad port 30a, 32a of the present embodiment further includes a relatively short tubing segment 48a which has a proximal end positioned at the periphery of the bladder 34a and a distal end extending away from the bladder 34a. The proximal end of the short tubing segment 48a extends into the interior of the bladder 34a and is preferably fixably and permanently joined with the bladder 34a at the periphery. The proximal end is constructed to resist occlusion from kinking in the region where the proximal end is joined with the bladder 34a. The first and second port couplings 38a, 40a are positioned across the open distal ends of the short tubing segments 48a of the first and second pad ports 30a, 32a, respectively, and are likewise preferably fixably and permanently joined with the distal ends. The short tubing segments 48a facilitate connection of the first and second port couplings 38a, 40a with the first and second line couplings 42, 44 and extend the heat transfer pad 12a away from the joint 46 to shield the patient from the relatively rigid joint 46 during operation of the system 10a. The short tubing segments 48a are preferably formed from the same flexible tubing and have essentially the same inside diameter as the first and second fluid circulation lines 16, 18. An insulating port sheath 50a(shown partially cut away) having a similar composition and construction to the line sheath 21 extends between the joint 46 and the heat transfer pad 12a and covers the short tubing segments 48a.

Although not shown, the first and second pad ports 30a, 32a are alternatively configured with a relatively long tubing segment, which is substituted for the short tubing segment 48a, thereby more distally positioning the first and second port couplings 38a, 40a. In yet another alternative, the first and second pad ports 30a, 32a are configured without any tubing segments. In accordance with this alternative, the first and second port couplings 38a, 40a are positioned at the periphery of the bladder 34a and are permanently affixed directly to the bladder 34a.

Heat transfer pads of the type having utility in the non-ambient temperature therapy system 10a are disclosed in commonly-owned U.S. Pat. Nos. 5,417,720 and 5,662,695 and U.S. Design Pats. Des. 348,106 and Des. 345,609, all of which are incorporated herein by reference. The heat transfer pads disclosed in the above-referenced patents and other like heat transfer pads having utility in the system 10a are commercially available from Breg, Inc., 2611 Commerce Way, Vista, Cailf. 92081, U.S.A. and are further disclosed at www.breg.com.

The pump 20 of the non-ambient temperature therapy system 10a is generally a means for driving the non-ambient temperature heat transfer fluid from the fluid reservoir 14 to the bladder 34a via the first fluid circulation line 16 and the first pad port 30a. The pump 20 is further a means for circulating the non-ambient temperature heat transfer fluid through the internal flowpath of the bladder 34a and for driving the heat transfer fluid from the bladder 34a to the fluid reservoir 14 via the second pad port 32a and second fluid circulation line 18.

The pump 20 of the non-ambient temperature therapy system 10a is not specific to any one structure or mechanism of operation, but can be selected from a number of pumps having differing structures and mechanisms of operation. For example, the pump 20 can inter alia be an axial pump, a centrifugal pump, a gear pump, or a reciprocating pump, each of which has its own distinct structure and mechanism of operation. Nevertheless, the pump 20 of the non-ambient temperature therapy system 10a is preferably operationally characterized as having a maximum pump output when the pump motor is at a set operating speed. Maximum pump output is defined herein as the fluid flow rate at the outlet of the pump when the pump motor is fixed at the set operating speed and the pump is pumping the given fluid against a minimal resistance to flow downstream of the pump. A minimal resistance to flow is typically experienced when the pump is only pumping against ambient atmospheric pressure at the pump outlet (i.e., essentially zero head pressure) and there are no other impediments to flow at or downstream of the pump outlet. A typical maximum pump output is on the order of about 200 ml/min.

The values of the set operating speed and maximum pump output are optimal values which often exceed actual values of pump operating parameters during system operation. In practice, the actual operating speed and actual pump output only match the set operating speed and maximum pump output, respectively, in the limited optimal case where there is minimal resistance to flow downstream of the pump. More commonly, there is an increased resistance to flow downstream of the pump exceeding the minimal resistance to flow, which causes a decrease in the actual operating speed of the pump motor below the set operating speed and a corresponding decrease in the actual pump output below the maximum pump output.

Thus, actual operating speed and correspondingly actual pump output are inversely correlated with resistance to flow of the system flowpath, within which the heat transfer fluid is circulated. In the case of the non-ambient temperature therapy system 10a, the system flowpath is an essentially closed loop from the fluid reservoir 14 to the heat transfer pad 12a and back to the fluid reservoir 14. A system designer specifies the pump 20 as a function of the system resistance to flow. In particular, a pump is selected for the non-ambient temperature therapy system 10a which includes a pump motor having sufficient power to drive the heat transfer fluid through the entire system flowpath at an acceptable flow rate (i.e., the actual pump output) against the system resistance to flow.

There are any number of ways for determining the system resistance to flow. Since the resistance to flow is correlated to the head pressure of the system and the pressure drop across the system, the system designer can calculate, measure, estimate, or otherwise determine the head pressure or pressure drop for the system as a whole or for individual components within the system flowpath and use this data to determine the system resistance to flow. An exemplary actual pump output is at least 100 ml/min at a head pressure of about 6.5 psi when the maximum pump output is 200 ml/min at a head pressure of about 0 psi.

A preferred pump satisfying the above-recited criteria is a pump having a single-speed pump motor. Such a pump is termed a single-speed pump and is defined herein as a pump having a pump motor which is permanently fixed at one set operating speed when pumping against a minimal downstream resistance to flow. The single-speed pump lacks means for the user to adjust or reset the set operating speed of the pump motor. As such, the set operating speed of the pump motor and correspondingly the maximum output of the single-speed pump are fixed by the manufacturer of the pump at the time of manufacture. Nevertheless, as noted above, the actual operating speed and actual pump output vary as a function of system resistance to flow.

An exemplary pump having utility in the system 10a is a single-speed submersible axial pump driven by a dc-powered electric motor, such as generally disclosed in U.S. Pat. No. 5,241,951, which is incorporated herein by reference. The system 10a employs a transformer 52 upstream of the pump 20 which converts ac power from a conventional ac wall outlet 54 to dc power. A power line 56 conveys the dc power from the transformer 52 to a dc motor in the pump 20. The power line 56 is exposed as it extends between the transformer 52 and a power connector 58 mounted on the first and second fluid circulation lines 16, 18. The power line 56 is enclosed within the line sheath 21 parallel to the first and second fluid circulation lines 16, 18 (shown in FIG. 2) as the power line 56 extends from the power connector 58 to the pump 20.

Alternatively, the dc-powered pump 20 obtains dc power directly from a dc power source, such as an automobile battery or a portable external or internal battery pack consisting of one or more disposable dry cell batteries or rechargeable batteries. In another alternative, the pump is driven by an ac-powered electric motor which is directly connected to the ac wall outlet 54.

An alternate pump satisfying the above-recited criteria is a pump having a variable-speed pump motor. Such a pump is termed a variable-speed pump and is defined herein as a pump which includes means for the user to actively vary the set operating speed of the pump motor. If a variable-speed pump is employed in the non-ambient temperature therapy system 10a in accordance with the preferred method of operation described hereafter, the user fixes the set operating speed of the pump motor in correspondence with a desired maximum output before initiating operation of the system 10a. Once operation of the system 10a is initiated, the user does not actively reset the set operating speed of the pump motor away from the initial set operating speed for the duration of the operating segment, which is defined as a time period when the system 10a is in continuous uninterrupted operation.

The distal ends of the first and second fluid circulation lines 16, 18 are attached to the pump 20 and open into an internal pumping chamber (not shown) of the pump 20. The pumping chamber and correspondingly the distal ends of the first and second fluid circulation lines 16, 18 are open to the surrounding environment 28. The pump 20 and distal ends of the first and second fluid circulation lines 16, 18 are operatively positioned in the interior of the fluid reservoir 14 when the non-ambient temperature therapy system 10a is in the assembled state, although the fluid reservoir 14 is not physically connected to (i.e., is structurally independent from) the pump 20 and the first and second fluid circulation lines 16, 18. As a result, operative positioning of the pump 20 and distal ends of the first and second fluid circulation lines 16, 18 places the first and second fluid circulation lines 16, 18 in fluid communication with the interior of the fluid reservoir 14. Although not shown, it is alternatively within the scope of the present invention to structurally integrate or otherwise physically connect the fluid reservoir 14 with the pump 20 and/or with the first and second fluid circulation lines 16, 18.

The effectiveness of the non-ambient temperature therapy system 10a in treating a desired region of the body of a patient is largely dependent on the pad temperature which is termed herein the system treatment temperature (TTi₁₀a). The treatment temperature is characterized as the surface temperature of the heat transfer pad 12a during system operation. There are a number of functional relationships between the treatment temperature and the system operating parameters. The functional relationship between the treatment temperature and the residence time of the heat transfer fluid in the pad (i.e, the pad residence time) is of particular interest. Specifically, the treatment temperature is directly correlated with the pad residence time for a low temperature treatment, while the treatment temperature is indirectly correlated with the pad residence time for a high temperature treatment.

The functional relationships between the resistance to flow of the system flowpath (i.e., the system resistance to flow) and the system operating parameters are also noteworthy. In particular, the system resistance to flow is indirectly correlated with the actual operating speed of the pump motor, the actual pump output, and the heat transfer fluid flow rate through the heat transfer pad 12a (i.e., the pad flow rate). Since the pad residence time is likewise indirectly correlated with the pad flow rate, it follows that the pad residence time is directly correlated with the system resistance to flow. Thus, the treatment temperature is directly correlated with the system resistance to flow during a low temperature treatment, i.e., the treatment temperature increases in response to an increase in the system resistance to flow and decreases in response to a decrease in the system resistance to flow. In contrast, the treatment temperature is indirectly correlated with the system resistance to flow during a high temperature treatment, i.e., the treatment temperature increases in response to a decrease in the system resistance to flow and decreases in response to an increase in the system resistance to flow. The present invention recognizes the above-recited functional relationship between treatment temperature and system resistance to flow. In practice, the present invention automatically imposes a desired treatment temperature on the non-ambient temperature therapy system 10a by employing a system flowpath configuration having a system resistance to flow which is specifically correlated with the desired treatment temperature.

The system flowpath, which circulates the heat transfer fluid from the fluid reservoir 14 to the heat transfer pad 12a and back to the fluid reservoir 14, generally comprises in series the first fluid circulation line 16, the heat transfer pad 12a, and the second fluid circulation line 18. The segment of the system flowpath extending through the heat transfer pad 12a is specifically termed the pad flowpath and comprises in series the first pad port 30a, the internal flowpath of the bladder 34a, and the second pad port 32a. Each component of the system flowpath has a resistance to flow of the heat transfer fluid therethrough, which is a function of many variables relating to the fixed (i.e., inherent) physical geometry of the structural component, including interalia the length and inside diameter of the component and the smoothness and composition of the inside walls of the component. These resistances to flow are termed individual inherent resistances to flow.

The system flowpath has an overall system inherent resistance to flow (RTF₁₀ₐ), which is defined as the sum of the individual inherent resistances to flow of each component within the system flowpath. Thus, the system inherent resistance to flow is generally the sum of the individual inherent resistance to flow of the first fluid circulation line 16 (RTF₁₆), the individual inherent resistance to flow of the heat transfer pad (RTF₁₂ₐ), and the individual inherent resistance to flow of the second fluid circulation line 18 (RTF₁₈), wherein the individual inherent resistance to flow of the heat transfer pad (RTF₁₂ₐ) is the sum of the individual inherent resistance to flow of the first pad port (RTF₃₀ₐ), the individual inherent resistance to flow of the internal flowpath of the bladder 34a (RTF₃₄ₐ), and the individual inherent resistance to flow of the second pad port 32a (RTF₃₂ₐ).

The system inherent resistance to flow for the system flowpath is expressed by equations (1) and (2) below:
RTF₁₀ₐ=RTF₁₆+RTF₁₂ₐ+RTF₁₈, wherein (1)
RTF₁₂ₐ=RTF₃₀ₐ+RTF₃₄ₐ+RTF₃₂ₐ. (2)
It is apparent from equations (1) and (2) that the system inherent resistance to flow can be adjusted by modifying the configuration of any one or more components within the system flowpath, thereby modifying the individual resistance to flow of the reconfigured component and correspondingly modifying the system resistance to flow of the entire non-ambient temperature therapy system 10a.

A preferred embodiment of the non-ambient temperature therapy system 10a is specific to low temperature treatment applications. In accordance with this embodiment, the non-ambient temperature therapy system 10a is provided with a pad flowpath adjustment member 60a which reconfigures the pad flowpath to increase the individual resistance to flow of the heat transfer fluid pad 12a and correspondingly to increase the overall system resistance to flow. As such, the non-ambient temperature therapy system 10a has a system adjusted resistance to flow (ARTF₁₀ₐ) which is greater than the system inherent resistance to flow (i.e., the resistance to flow of the system 10a in the absence of the pad flowpath adjustment member 60a). Inclusion of the pad flowpath adjustment member 60a, which adjusts the resistance to flow of the non-ambient temperature therapy system 10a, enables the system 10a to achieve a desired treatment temperature, which is greater than what the treatment temperature would otherwise be in the absence of the reconfigured pad flowpath. The degree to which the desired treatment temperature is increased is a function of the degree to which the system resistance to flow is increased by the pad flowpath adjustment member 60a. Determination of this function is readily within the purview of the skilled artisan applying the teaching herein.

The pad flowpath adjustment member 60a is mounted in the pad flowpath of the heat transfer pad 12a and, more particularly, is mounted either in the internal flowpath of the bladder 34a, in the first pad port 30a which includes the first port coupler 38a, or in the second pad port 32a which includes the second port coupler 40a. In a preferred embodiment, the pad flowpath adjustment member 60a is mounted in the second pad port 32a. The term “mounted in” as used herein broadly encompasses one of the following alternatives: 1) positioning the pad flowpath adjustment member 60a internally within a component of the pad flowpath; serially attaching the pad flowpath adjustment member 60a to a component of the pad flowpath; or 3) integrating the pad flowpath adjustment member 60a into the construct of a component of the pad flowpath.

The pad flowpath adjustment member 60a is preferably an in-line passive device mounted in the first pad port 30a or second pad port 32a of the pad flowpath and most preferably in the second pad port 32a. In any case, the pad flowpath adjustment member 60a is structurally distinct from the first or second pad port 30a, 32a. The in-line passive device is inserted directly into the first or second pad port 30a, 32a (and most preferably in the second pad port 32a) to increase the resistance to flow of the pad flowpath and reduce the pad flow rate when the speed of the pump motor is fixed at the set operating speed and the pump 20 is correspondingly at the maximum pump output. The term “passive device” as used herein characterizes a device which is not user adjustable.

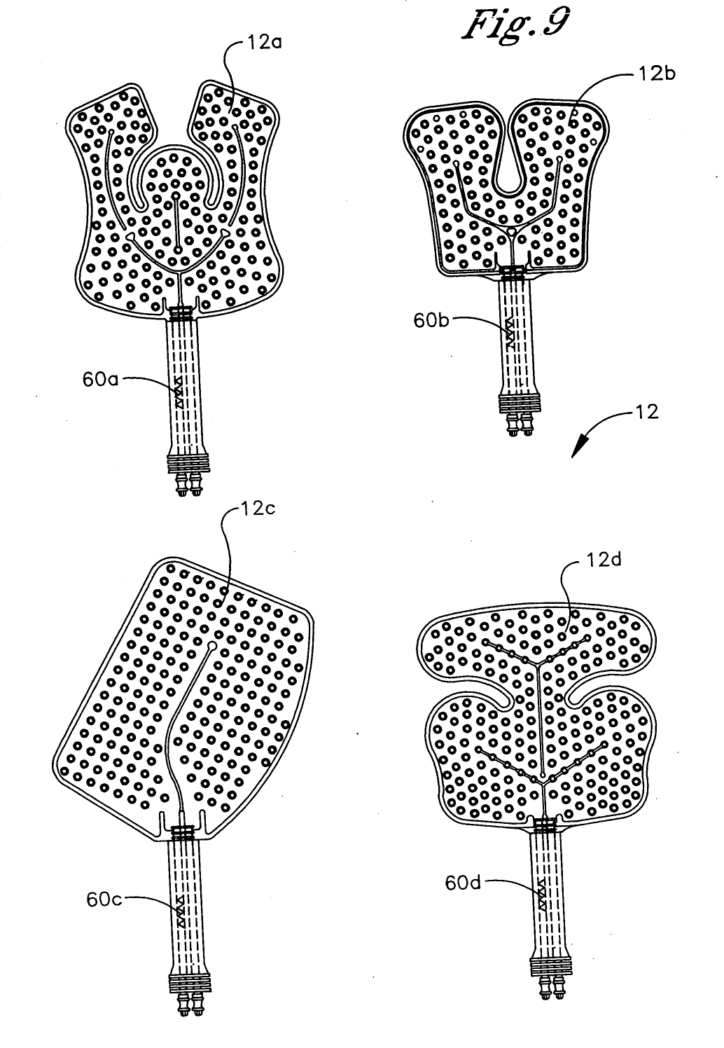

An exemplary passive device which functions as the pad flowpath adjustment member 60a is a baffle. The baffle redirects the heat transfer fluid as it passes through the pad flowpath past the baffle. A preferred baffle 60a is a threaded rod shown in FIG. 4 which has a continuous screw thread 62 with multiple spiraled turns about a cylindrical core 64 in the manner of a conventional screw. The threaded rod 60a is coaxially positioned within the short tubing segment 48a of the first or second pad port 30a, 32a (and most preferably the second pad port 32a as shown in FIG. 3). The thread 62 has an outside diameter which is essentially equal to the inside diameter of the short tubing segment 48a so that essentially all of the heat transfer fluid follows the spiral flowpath around the cylindrical core 64 of the threaded rod 60a, while essentially none of the heat transfer fluid passes between the outer periphery of the thread 62 and the inner wall of the short tubing segment 48a. Alternatively, the passive device 60a is a plate as shown in FIGS. 5 and 6 or a nozzle as shown in FIGS. 7 and 8. The plate or nozzle 60a is positioned in the short-tubing segment 48a of the first or second pad port 30a, 32a (and most preferably the second pad port 32a) and each has an orifice 66 for fluid flow therethrough with a reduced cross-sectional area relative to the short tubing segment 48a.

An example of a pad flowpath adjustment member 60a which is integrated into the construct of a component of the pad flowpath is a crimp formed in the wall of the short tubing segment 48a of the first or second pad port 30a, 32a (and most preferably in the second pad port 32a), which reduces the cross-sectional area of the short tubing segment 48a at the point of the crimp. An alternate integrated pad flowpath adjustment member 60a is provided by replacing all or a portion of the short tubing segment 48a of the first or second pad port 30a, 32a (and most preferably in the second pad port 32a) with a tubing segment having a reduced cross-sectional area relative to the remaining short tubing segment 48a.

The system adjusted resistance to flow for the system flowpath of the system 10a (ARTF₁₀ₐ) is expressed by equations (3) and (4) below:
ARTF₁₀ₐ=RTF₁₆+ARTF₁₂ₐ+RTF₁₈, wherein (3)
ARTF₁₂ₐ=ARTF₃₀ₐ+RTF₃₄ₐ+RTF₃₂ₐ; (4a)
ARTF₁₂ₐ=RTF₃₀ₐ+ARTF₃₄ₐ+RTF₃₂ₐ; or (4b)
ARTF₁₂ₐ=RTF₃₀ₐ+RTF₃₄ₐ+ARTF₃₂ₐ. (4c)
Equation (4a) represents the case where the pad flowpath adjustment member 60a is mounted in the internal flowpath of the bladder 34a, so that the bladder 34a has an adjusted resistance to flow (ARTF₃₄ₐ) which is great than the individual inherent resistance to flow of the bladder 34a. Equation (4b) represents the case where the pad flowpath adjustment member 60a is mounted in the first pad port 30a, so that the first pad port 30a has an adjusted resistance to flow (ARTF₃₀ₐ) which is great than the individual inherent resistance to flow of the first pad port 30a. Equation (4c) represents the most preferred case where the pad flowpath adjustment member 60a is mounted in the second pad port 32a, so that the second pad port 32a has an adjusted resistance to flow (ARTF₃₂ₐ) which is great than the individual inherent resistance to flow of the second pad port 32a.

A system back pressure flow restrictor (not shown) is optionally mounted in-line along the length of the second fluid circulation line 18 downstream of the heat transfer pad 12a , although in many cases the pad flowpath adjustment member 60a obviates the need for the back pressure flow restrictor. In the event the system 10a lacks sufficient fluid back pressure downstream of the heat transfer pad 12a, the optional back pressure flow restrictor creates sufficient fluid back pressure to maintain the bladder 34a properly inflated during the preferred method of operating the non-ambient temperature therapy system 10a. The back pressure flow restrictor is an in-line passive device such as described or is alternately a variable in-line device such as the adjustable flow restrictor valve disclosed in U.S. Pat. No. 5,241,951. However, once operation of the system 10a is initiated, the user does not vary the variable back pressure flow restrictor away from the initial setting so that the individual inherent resistance to flow of the second fluid circulation line 18 remains constant for the entire operating segment.

Referring additionally to the FIG. 9, the heat transfer pad 12a of the non-ambient temperature therapy system 10a is optionally a member of a pad family generally designated 12. In addition to the heat transfer pad 12a, the pad family 12 comprises heat transfer pads 12b, 12c, 12d. Each heat transfer pad 12a, 12b, 12c, 12d preferably has a substantially different geometry than the other, i.e., a different shape and/or one or more different dimensions of size, e.g., length, width, etc. The specific number of individual heat transfer pads and the specific geometry of each heat transfer pad shown in the particular pad family 12 of FIG. 9 are selected solely for purposes of illustration and are not intended to limit the present invention. In practice the pad family 12 can comprise essentially any number of individual heat transfer pads and each individual heat transfer pad can have essentially any geometry within the limits of practicality.

It is apparent from FIG. 2 that the non-ambient temperature therapy system of the present invention can accommodate only one heat transfer pad from the pad family 12 at any given time in the assembled state. FIGS. 1 and 2 illustrate one embodiment of the non-ambient temperature therapy system, wherein the system 10a includes the heat transfer pad 12a. It is alternately within the scope of the present invention to substitute any one of the remaining heat transfer pads 12b, 12c, or 12d in the pad family 12 for the heat transfer pad 12a at the discretion of a user when assembling the non-ambient temperature therapy system. Thus, an alternate embodiment of the non-ambient temperature therapy system substitutes the heat transfer pad 12b for the heat transfer pad 12a. The resulting system referred to hereafter as 10b (although not shown) comprises the same fluid reservoir 14, first and second fluid circulation lines 16, 18 and pump 20 as the system 10a as well as the different heat transfer pad 12b. Another alternate embodiment of the non-ambient temperature therapy system referred to as 10c (not shown) differs from the system 10a only in the substitution of the heat transfer pad 12c for the heat transfer pad 12a. Still another alternate embodiment of the non-ambient temperature therapy system referred to as 10d (not shown) differs from the system 10a only in the substitution of the heat transfer pad 12d for the heat transfer pad 12a.

The description of the non-ambient temperature therapy system 10a recited above applies generally to each of the remaining alternate non-ambient temperature therapy systems 10b, 10c, 10d, except for the differences between the particular heat transfer pads 12a, 12b, 12c, or 12d employed therein. Furthermore, the general construction of all the heat transfer pads 12a, 12b, 12c, 12d in the pad family 12 is preferably essentially the same apart from apparent differences in the geometry of the individual heat transfer pads. As such, the construction of the heat transfer pad 12a described above applies generally to each of the remaining heat transfer pads 12b, 12c, 12d in the pad family 12. Although the heat transfer pads 12a, 12b, 12c, 12d all have different geometries, their individual inherent resistances to flow are all essentially equal to one another and each exhibits essentially the same heat transfer rate between the heat transfer pad and the body of a patient for a given area of the heat transfer pad.

The overall system inherent resistance to flow for the system flowpath of each system 10b, 10c, 10d (RTF_{10b}, RTF_{10c}, RTF_{10d}) and their relation to one another and system 10a is expressed by equations (5)-(11) below:
RTF_{10b}=RTF₁₆+RTF_{12b}+RTF₁₈, wherein (5)
RTF_{12b}=RTF_{30b}+RTF_{34b}+RTF_{32b} (6)
RTF_{10c}=RTF₁₆+RTF_{12c}+RTF₁₈, wherein (7)
RTF_{12c}=RTF_{30c}+RTF_{34c}+RTF_{32c} (8)
RTF_{10d}=RTF₁₆+RTF_{12d}+RTF₁₈, wherein (9)
RTF_{12d}=RTF_{30d}+RTF_{34d}+RTF_{32d} (10)
RTF₁₀ₐ=RTF_{10b}=RTF_{10d}=RTF_{10d} (11)

As with the heat transfer pad 12a, the remaining heat transfer pads 12b, 12c, 12d in the pad family 12 are each preferably provided with a pad flowpath adjustment member 60b, a pad flowpath adjustment member 60c, and a pad flowpath adjustment member 60d, respectively (none are shown). As in the case of the pad flowpath adjustment member 60a, the pad flowpath adjustment member 60b provides the system 10b with a system adjusted resistance to flow (ARTF10b), the pad flowpath adjustment member 60c provides the system 10c with a system adjusted resistance to flow (ARTF_{10c}), and the pad flowpath adjustment member 60d provides the system 10d with a system adjusted resistance to flow (ARTF_{10d}). The pad flowpath adjustment members 60b, 60c, 60d all have an essentially similar construction to pad flowpath adjustment member 60a described above although two or more of the pad flowpath adjustment members may have different individual resistances to flow as described below.

The system adjusted resistance to flow for the system flowpath of each alternate system 10b, 10c, 10d is expressed by equations (12)-(17) below:
ARTF_{10b}=RTF₁₆+ARTF_{12b}+RTF₁₈, wherein (12)
ARTF_{12b}=ARTF_{30b}+RTF_{34b}+RTF_{32b}; (13a)
ARTF_{12b}=RTF_{30b}+ARTF_{34b}+RTF_{32b}; or (13b)
ARTF_{12b}=RTF_{30b}+RTF_{34b}+ARTF_{32b}. (13c)
ARTF_{10c}=RTF₁₆+ARTF_{12c}+RTF₁₈, wherein (14)
ARTF_{12c}=ARTF_{30c}+RTF₃₄+RTF_{32c}; (15a)
ARTF_{12c}=RTF_{30c}+ARTF_{34c}+RTF_{32c}; or (15b)
ARTF_{12c}=RTF_{30c}+RTF₃₄+ARTF_{32c}, (15c)
ARTF_{10d}=RTF₁₆+ARTF_{12d}+RTF₁₈, wherein (16)
ARTF_{12d}=ARTF_{30d}+RTF_{34d}+RTF_{32d}; or (17a)
ARTF_{12d}=RTF_{30d}+ARTF_{34d}+RTF_{32d}; or (17b)
ARTF_{12d}=RTF_{30d}+RTF_{34d}+ARTF_{32d}. (17c)

In accordance with one embodiment of the present invention, the pad flowpath adjustment members are selected such that each pad flowpath adjustment member 60a, 60b, 60c, 60d imparts an adjusted resistance to flow to its respective heat transfer pad 12a, 12b, 12c, 12d which is different than one or more of the other heat transfer pads. Since the non-ambient temperature therapy systems 10a, 10b, 10c, 10d are essentially identical in all other respects, the system adjusted resistance to flow of each non-ambient temperature therapy system 10a, 10b, 10c, 10d differs from the others only as a function of the respective pad flowpath adjustment members 60a, 60b, 60c, 60d. Accordingly, the system designer pairs the pad flowpath adjustment members with the heat transfer pads such that each heat transfer pad imparts a fixed predetermined system adjusted resistance to flow and correspondingly a fixed desired treatment temperature to its respective assembled system. Consequently, each non-ambient temperature therapy system provides a desired treatment temperature during system operation which is specific to its respective heat transfer pad. In particular, the system 10a provides the fixed desired treatment temperature (TT₁₀ₐ), the system 10b provides the fixed desired treatment temperature (TT_{10b}), the system 10c provides the fixed desired treatment temperature (TT_{10c}), and the system 10d provides the fixed desired treatment temperature (TT_{10d}). Some or all of the above-recited fixed desired treatment temperatures are preferably different from one another.

The geometry of each heat transfer pad 12a, 12b, 12c, 12d is often dictated by the shape of a particular region of the body being treated so that the heat transfer pad exhibits close-fitting conformance to the contours of the specified region. For example, the heat transfer pad 12a is designed to conform to the knee or shoulder, the heat transfer pad 12b is designed to conform to the ankle, the heat transfer pad 12c is designed to conform to the back, while the heat transfer pad 12d is designed for general conformance to non-specific regions of the body. The desired treatment temperature is also dictated by the region of the body patient being treated because certain regions of the body are more sensitive and less able to withstand non-ambient temperature extremes than other regions. For example, the desired treatment temperature for the knee or shoulder is generally lower than the desired treatment temperature for the ankle during a low temperature treatment.

In view of the above, the system designer specifies a pad flowpath adjustment member 60a for mounting in the heat transfer pad 12a specific to knee or shoulder treatment applications which has a relatively low resistance to flow, thereby achieving a lower system adjusted resistance to flow and effecting a lower desired treatment temperature. In contrast, the system designer specifies a pad flowpath adjustment member 60b for mounting in the heat transfer pad 12b specific to ankle treatment applications which has a relatively high resistance to flow, thereby achieving a higher system adjusted resistance to flow and effecting a higher desired treatment temperature. In any case, the desired treatment temperature is preferably in a range of about 45 to 55° F. for a low temperature treatment.

The relations between the above-recited parameters for the systems 10a, 10c in accordance with the present example are expressed by the equations (18) and (19) below:
ARTF₁₀ₐ<ARTF_{10c} (18)
TT₁₂ₐ<TT_{12c} (19)
In sum, the individual adjusted resistances to flow of the heat transfer pads 12a, 12b, 12c, 12d and correspondingly the system adjusted resistances to flow and the desired treatment temperatures of the non-ambient temperature therapy systems 10a, 10b, 10c, 10d are fixed to correspond with the specific requirements of the desired treatment application.

### Method of Operation

An embodiment of a method of operating the non-ambient temperature therapy system 10a is described hereafter with further reference to the drawings. It is understood that the operating method described hereafter applies generally to operation of the non-ambient temperature therapy systems 10b, 10c, 10d as well. The method is initiated by a series of set-up steps, wherein the heat transfer pad 12a is selected by the user as a function of the desired treatment application for a knee or shoulder treatment application. The heat transfer pad 12a is then mounted on the skin of the desired body region of the patient where non-ambient temperature treatment is desired, which in the present case is the knee 68. An additional thin padding material, such as a soft cloth, may be placed on the skin between the bladder 34a and the skin for the comfort of the patient.

Mounting the heat transfer pad 12a on the knee 68 is typically effected by wraps (not shown) which are permanently attached to the heat transfer pad 12a or which are separately provided. The configuration of the heat transfer pad 12a renders it readily and fully conformable to the contours of the knee 68 of the patient on which it is mounted. A plurality of slits 70 are formed in the heat transfer pad 12a which enhance conformance to the knee 68. The configuration of the heat transfer pad 12a enables a large fraction of the pad surface area to contact the knee 68 and advantageously facilitates heat transfer between the heat transfer pad 12a and the knee 68.

A charge of heat transfer fluid is placed in the fluid reservoir 14 via the opening 24. The heat transfer fluid is preferably a liquid and the volume of the heat transfer fluid charge is preferably several times the volume of the heat transfer pad 12a. A preferred heat transfer fluid for a low temperature treatment is cold water. Solid ice can also be charged to the fluid reservoir 14 with the cold water to produce ice water for the low temperature treatment. A preferred heat transfer fluid for a high temperature treatment is hot water. The charged fluid reservoir 14 is positioned proximal the patient and the pump 20 is submersed in the heat transfer fluid within the fluid reservoir 14. The lid 26 is placed over the opening 24 with the first and second circulation lines 16, 18 extending from the fluid reservoir 14. The power line 56 of the pump 20 is connected to a source of electrical power. Once the heat transfer pad 12a is mounted on the knee 68 and the pump 20 is operatively positioned, the couplings 38a and 42 and the couplings 40a and 44, respectively, are locked together to close the joint 46 and complete the set-up steps. The resulting non-ambient temperature therapy system 10a is in an assembled state and in a condition for operation.

Operation of the system 10a is initiated by powering up the pump 20, preferably by means of an on/off power switch (not shown) mounted on the power connector 58 or simply by connecting the power line 56 with the wall outlet 54 and the power connector 58. The pump 20 withdraws the heat transfer fluid from the fluid reservoir 14 and drives the fluid through the first fluid circulation line 16 into the heat transfer pad 12a, thereby inflating the bladder 34a. The pump 20 circulates the heat transfer fluid through the internal flowpath of the bladder 34a, causing the temperature of the heat transfer pad 12a to drop to the desired treatment temperature in the case of a low temperature treatment and in turn causing the transfer of body heat from the patient to the pad 12a by a heat transfer mechanism. In the case of a high temperature treatment, the heat transfer fluid causes the temperature of the heat transfer pad 12a to rise to the desired treatment temperature and in turn causes heat to be transferred from the pad 12a to the body of the patient by a heat transfer mechanism.

In either case, the heat transfer fluid is withdrawn from the heat transfer pad 12a after circulating therethrough and is returned to the fluid reservoir 14. The withdrawn heat transfer fluid mixes with the relatively large volume of heat transfer fluid remaining in the heat transfer reservoir 14, which functions as a heat sink, essentially reversing any temperature increase or decrease in the withdrawn heat transfer fluid due to heat transfer with the body. The pump 20 is operated continuously as long as treatment is desired, thereby providing continuous steady-state circulation of the heat transfer fluid between the fluid reservoir 14 and heat transfer pad 12a for the duration of the treatment. Operation of the system 10a is terminated by simply shutting off the electrical power to the pump 20.

While the forgoing preferred embodiments of the invention have been described and shown, it is understood that alternatives and modifications, such as those suggested and others, may be made thereto and fall within the scope of the invention.

We claim:

1. A therapy system for therapeutically treating a desired region of the body comprising:
a fluid reservoir for storing a charge of a heat transfer fluid at a non-ambient temperature;
a heat transfer pad conformable to a body region having an ambient temperature, said heat transfer pad including a first pad port, a bladder, and a second pad port and defining a pad flowpath extending in series therethrough;
a first fluid circulation line connectable to said first pad port and in fluid communication with said fluid reservoir for withdrawing said heat transfer fluid from said fluid reservoir and delivering said heat transfer fluid to said heat transfer pad via said first pad port;
a second fluid circulation line connectable to said second pad port and in fluid communication with said fluid reservoir for withdrawing said heat transfer fluid from said heat transfer pad via said second pad port and delivering said heat transfer fluid to said fluid reservoir, wherein said first fluid circulation line, said pad flowpath, and said second fluid circulation line define a system flowpath extending in series therethrough;
a pump including a motor fixed at a set operating speed to define a maximum pump output, wherein said pump is in fluid communication with said fluid reservoir and said first fluid circulation line for driving said heat transfer fluid through said system flowpath against a system resistance to flow; and
a pad flowpath adjustment member positioned in said pad flowpath to adjust said system resistance to flow to an adjusted system resistance to flow, said adjusted system resistance to flow fixing a desired treatment temperature of said heat transfer pad correlated to said body region in response to said heat transfer fluid circulating through said pad flowpath.

2. The therapy system of claim 1, wherein said non-ambient temperature is a lower temperature than said ambient temperature of said body region.

3. The therapy system of claim 1, wherein said non-ambient temperature is a higher temperature than said ambient temperature of said body region.

4. The therapy system of claim 1, wherein said adjusted system resistance to flow is greater than said system resistance to flow.

5. The therapy system of claim 1, wherein said desired treatment temperature is indirectly correlated with said adjusted system resistance to flow.

6. The therapy system of claim 1, wherein said pump operates at an actual pump output less than said maximum pump output.

7. The therapy system of claim 1, wherein said pad flowpath adjustment member is mounted in said second pad port.

8. The therapy system of claim 1, wherein said pad flowpath adjustment member is a crimp formed in a wall of said first or second pad port.

9. The therapy system of claim 1, wherein said pad flowpath adjustment member is a threaded rod positioned in said first or second pad port.

10. The therapy system of claim 1, wherein said pad flowpath adjustment member is a plate or nozzle having an aperture formed therethrough positioned in said first or second pad port.

11. The therapy system of claim 1, wherein said pad flowpath adjustment member is a tubing segment of said second pad port having a reduced cross-sectional area relative to said first pad port.

12. The therapy system of claim 1, wherein said pad flowpath adjustment member is a tubing segment of said first pad port having a reduced cross-sectional area relative to said second pad port.

13. A therapy system for therapeutically treating a desired region of the body comprising:
a fluid reservoir for storing a charge of a heat transfer fluid at a non-ambient temperature;
a pad family including a first heat transfer pad having a first geometry and conformable to a first body region having an ambient temperature, said first heat transfer pad including a first pad port, a bladder, and a second pad port and defining a first pad flowpath extending in series therethrough, said pad family further including a second heat transfer pad having a second geometry different from said first geometry and conformable to a second body region having said ambient temperature, said second heat transfer pad including a first pad port, a bladder, and a second pad port and defining a second pad flowpath extending in series therethrough;
a first fluid circulation line connectable to said respective first pad port of said first or second heat transfer pad and in fluid communication with said fluid reservoir for withdrawing said heat transfer fluid from said fluid reservoir and delivering said heat transfer fluid to said first or second heat transfer pad via said respective first pad port;
a second fluid circulation line connectable to said respective second pad port of said first or second heat transfer pad and in fluid communication with said fluid reservoir for withdrawing said heat transfer fluid from said first or second heat transfer pad via said respective second pad port and delivering said heat transfer fluid to said fluid reservoir, wherein said first fluid circulation line, said first pad flowpath, and said second fluid circulation line define a first system flowpath extending in series therethrough, and wherein said first fluid circulation line, said second pad flowpath, and said second fluid circulation line define a second system flowpath extending in series therethrough;
a pump including a motor fixed at a set operating speed to define a maximum pump output, wherein said pump is in fluid communication with said fluid reservoir and said first fluid circulation line for driving said heat transfer fluid through said first or second system flowpath against a system resistance to flow; and
a first pad flowpath adjustment member positioned in said first pad flowpath to adjust said system resistance to flow to a first adjusted system resistance to flow, said first adjusted system resistance to flow fixing a first desired treatment temperature of said first heat transfer pad correlated to said first body region in response to said heat transfer fluid circulating through said first pad flowpath; and
a second pad flowpath adjustment member positioned in said second pad flowpath to adjust said system resistance to flow to a second adjusted system resistance to flow, said second adjusted system resistance to flow fixing a second desired treatment temperature of said second heat transfer pad correlated to said second body region in response to said heat transfer fluid circulating through said second pad flowpath.

14. The therapy system of claim 13, wherein said non-ambient temperature is a lower temperature than said ambient temperature of said body region.

15. The therapy system of claim 13, wherein said second adjusted resistance to flow is less than said first adjusted system resistance to flow.

16. The therapy system of claim 13, wherein said second desired treatment temperature is less than said first desired treatment temperature.

17. The therapy system of claim 16, wherein said first body region is an ankle and said second body region is a knee.

18. The therapy system of claim 13, wherein said first and-second pad flowpath adjustment members are mounted in said respective second pad ports of said first and second heat transfer pads.

19. The therapy system of claim 13, wherein said first or second pad flowpath adjustment member is a threaded rod positioned in said first or second pad port of said first or second heat transfer pad.

20. A method for therapeutically treating a desired region of the body with a non-ambient temperature treatment fluid comprising:
storing a charge of a heat transfer fluid at a non-ambient temperature in a fluid reservoir;
positioning a heat transfer pad on a body region having an ambient temperature, said heat transfer pad including a first pad port, a bladder, and a second pad port and defining a pad flowpath extending in series therethrough;
connecting a first fluid circulation line in fluid communication with said fluid reservoir to said first pad port;
withdrawing said heat transfer fluid from said fluid reservoir and delivering said heat transfer fluid to said heat transfer pad via said first pad port;
connecting a second fluid circulation line in fluid communication with said fluid reservoir to said second pad port, wherein said first fluid circulation line, said pad flowpath, and said second fluid circulation line define a system flowpath extending in series therethrough;
withdrawing said heat transfer fluid from said heat transfer pad via said second pad port and delivering said heat transfer fluid to said fluid reservoir;
driving said heat transfer fluid through said system flowpath against a system resistance to flow with a pump, wherein said pump includes a motor fixed at a set operating speed defining a maximum pump output and said pump is in fluid communication with said fluid reservoir and said first fluid circulation line; and
positioning a pad flowpath adjustment member in said pad flowpath to adjust said system resistance to flow to an adjusted system resistance to flow, said adjusted system resistance to flow fixing a desired treatment temperature of said heat transfer pad correlated to said body region in response to said heat transfer fluid circulating through said pad flowpath.

21. The method of claim 20, wherein said body region is a first body region, said heat transfer pad is a first heat transfer pad, said pad flowpath is a first pad flowpath, said system flowpath is a first system flowpath, said pad flowpath adjustment member is a first pad flowpath adjustment member, said adjusted system resistance to flow is a first adjusted system resistance to flow, and said desired treatment temperature is a first desired treatment temperature, said method further comprising:
positioning a second heat transfer pad on a second body region having said ambient temperature, said second heat transfer pad including a first pad port, a bladder, and a second pad port and defining a second pad flowpath extending in series therethrough;
disconnecting said first fluid circulation line from said first pad port of said first heat transfer pad and reconnecting said first fluid circulation line to said first pad port of said second heat transfer pad;
withdrawing said heat transfer fluid from said fluid reservoir and delivering said heat transfer fluid to said second heat transfer pad via said first pad port of said second heat transfer pad;
disconnecting said second fluid circulation line from said second pad port of said second heat transfer pad and reconnecting said second fluid circulation line to said second pad port of said second heat transfer pad, wherein said first fluid circulation line, said second pad flowpath, and said second fluid circulation line define a second system flowpath extending in series therethrough;
withdrawing said heat transfer fluid from said second heat transfer pad via said second pad port of said second heat transfer pad and delivering said heat transfer fluid to said fluid reservoir;
driving said heat transfer fluid through said second system flowpath against said system resistance to flow with said pump; and
positioning a second pad flowpath adjustment member in said second pad flowpath to adjust said system resistance to flow to a second adjusted system resistance to flow, said second adjusted system resistance to flow fixing a second desired treatment temperature of said second heat transfer pad correlated to said second body region in response to said heat transfer fluid circulating through said second pad flowpath.

22. The method of claim 20, wherein said non-ambient temperature is a lower temperature than said ambient temperature of said body region.

23. The method of claim 21, wherein said second adjusted resistance to flow is less than said first adjusted system resistance to flow.

24. The method of claim 21, wherein said second desired treatment temperature is less than said first desired treatment temperature.

25. The method of claim 21, wherein said first body region is an ankle and said second body region is a knee.
